(19) United States
(12) Patent Application Publication
(10) Pub. No.: US 2025/0285560 A1
(43) Pub. Date: Sep. 11, 2025

(54) AR MARKER FOR INJECTION

(71) Applicant: NOBLE INTERNATIONAL, LLC, Orlando, FL (US)

(72) Inventors: Jeffery A. LETTMAN, Orlando, FL (US); Randy CARDOZA, Winter Garden, FL (US); Chris EVANS, Rehoboth Beach, DE (US)

(21) Appl. No.: 18/862,702

(22) PCT Filed: May 3, 2023
(86) PCT No.: PCT/US2023/020858

§ 371 Date: Nov. 4, 2024

Related U.S. Application Data
(60) Provisional application No. 63/337,796, filed on May 3, 2022.

(51) Int. Cl. G09B 23/28; A61M 5/42; G09B 5/06

(52) U.S. Cl. CPC G09B 23/285; A61M 5/422; A61M 5/425; A61M 5/427; G09B 5/065

(57) ABSTRACT

Disclosed is an AR self-injection training system and method of use incorporating a marker specifically designed for device-naive self-injection patients. The marker is designed so that it can be removably adhered to an injection site. Once in position it anchors AR content to the injection site for training. The marker is designed to associate AR content to the injection site for training and allows patient to leave the marker in place and inject to the same site. Providing a system that is able to identify a site of injection and to provide salient instructions streamlines the process of training and makes the transition to using a real-device relatively seamless. The provided embodiments can also incorporate error correction functionality and even local anesthesia or analgesic. Following training, the opening in the marker allows patients to leave the marker in place and inject to the same site.

### BACKGROUND

A concerning aspect related to injectable therapeutics relates to the user's ability to deliver an injection and deliver it effectively. Many injectable therapeutics are self-administered, and there is often a stigma with self-delivery of an injectable medication. Needle-anxiety and fear of failing to deliver a complete dose, inability to operate the dose-delivery device correctly, fear of accidental needle stick during manipulation of the dose delivery device, among other concerns, in users without any medical knowledge, or with medical training, can create a barrier to effective parenteral therapy. Furthermore, incorrect placement of the device on the target injection site on the user can prevent a user from obtaining an accurate dose of medicament, or can cause malfunctions in the device itself, resulting in noncompliance with the therapeutic regimen.

Augmented reality (AR) has been shown to positively affect mental workload and task performance across a broad range of application contexts. One study focused on training senior orthopedic surgical residents on the use of a new implant found that compared with traditional training methods, there was a 570% gain in efficiency in learning when training with an AR system. However, AR applications for self-injection training face some barriers. Depending on the drug, patients may be technologically naïve and injection surfaces are relatively small and non-uniform, making them a poor fit for new marker-less AR applications.

### SUMMARY

Disclosed is an AR self-injection training system incorporating a marker specifically designed for device-naïve self-injection patients. The marker is designed so that it can be removably adhered to an injection site. The marker is designed to associate AR content to the injection site for training and allows patients to leave the marker in place and inject to the same site. Providing a system that is able to identify a site of injection and to provide salient instructions streamlines the process of training and makes the transition to using a real-device relatively seamless. The provided embodiments can also incorporate error correction functionality and even local anesthesia or analgesic. Following training, the opening in the marker allows patients to leave the marker in place and inject to the same site.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows the marker adhered to an injection site and an example of the AR content interacting with the marker in digital space. The device detects and tracks the subject and marker using a sensor on the device.

FIG. 2 shows the subject performing an AR scenario. The AR content is viewed on a screen of a device running the AR component.

FIG. 3 shows the subject using the marker to self-administer a physical injection device.

FIG. 4 is a flow diagram representing exemplary steps in a method of using the AR training system.

FIG. 5 shows a perforated marker with integrated electronics.

FIG. 6 shows different versions of markers with different components. FIG. 6A shows a marker with an analgesic on the attaching side of the marker. FIG. 6B shows a cold component on an attaching side of the marker. FIG. 6C shows a vibrating component position on the attaching side of the marker.

### DEFINITIONS

For the purposes of promoting an understanding of the principles and operation of the

invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

The term “subject” as used herein refers to an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

The term “augmented reality” or “AR” as used herein refers to computer controlled sensory information presented to a user. AR may comprise visual, auditory, vestibular, haptic, gustatory, olfactory, pain, temperature, kinesthetic or other sensory stimulation, secondary activities, thought processes, visual thinking, verbal thinking, emotions, medications, chemicals, physiological manipulations, neurofeedback, psychotherapy, videoconferencing, video recordings, social interaction, virtual reality (“VR”), or guided imagery. Typically. AR will involve simulation of a real-life event or activity and will involve provision of information via one or multiple sensory modalities, including visual, auditory, haptic, somatosensory and/or olfactory.

The term “AR component” refers to any device that is capable of providing AR to a user. Examples of AR components may include a visual display. speaker, haptic devices including but not limited to gyroscope or vibrator component, temperature generator, pressure generator, and the like, and combinations thereof.

The terms “administering” or “administration” of an agent. drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

### DETAILED DESCRIPTION

Disclosed herein is an AR self-injection training system and method of use for addressing many common errors in patient use of self-injection devices. Some of these errors include failure to actuate the device with sufficient force to deliver an effective amount of medicament, or accidental actuation of the device prior to intended actuation. Other errors include use of sufficient force to satisfy actuation of the device, but insufficient force to quickly depress the plunger. Intimidation in self-administering medication, particularly via injection plays a large part in errors occurring during use of the self-injection devices. Additionally, when a self-injection device is used to administer medicament to a patient by another. it may be difficult to determine whether the dose of medicament is adequately delivered to the patient. Use of a training device to facilitate correct use of the drug delivery device will enhance compliance and ensure accurate dosage of medicament received.

AR content is not yet common in the self-injection training space. Many existing AR applications in healthcare locate on images in support material (pamphlets, packaging, etc.), or on virtual models. The unique feature of this approach is that the marker is designed so that the usage environment would be identical for the training material and the actual injection. It would also be possible to view training steps overlayed on top of the actual device in-situ.

In certain embodiments, the basic configuration of the AR training system consists of a marker, which has adhesive to removably bond to an injection site, and an opening to admit a self-injection device (i.e. autoinjector or PFS); and a smartphone, which is running software configured to position AR content on the marker; and optionally an injection device which is the subject of training.

In a specific embodiment, the marker is enhanced with pain alleviation through analgesic or topical anesthesia.

In certain embodiments, the marker contains one or more medical sensors. The medical sensors detect marker placement, temperature, strain, acceleration, or LED transmissibility.

In certain embodiments, the AR system incorporates error-correction during or after use. A further enhancement would be usage tracking using sensors on the smartphone. The smartphone could alternatively be a purpose-built AR device, like the Microsoft HoloLens, or Google Glass.

### System Overview

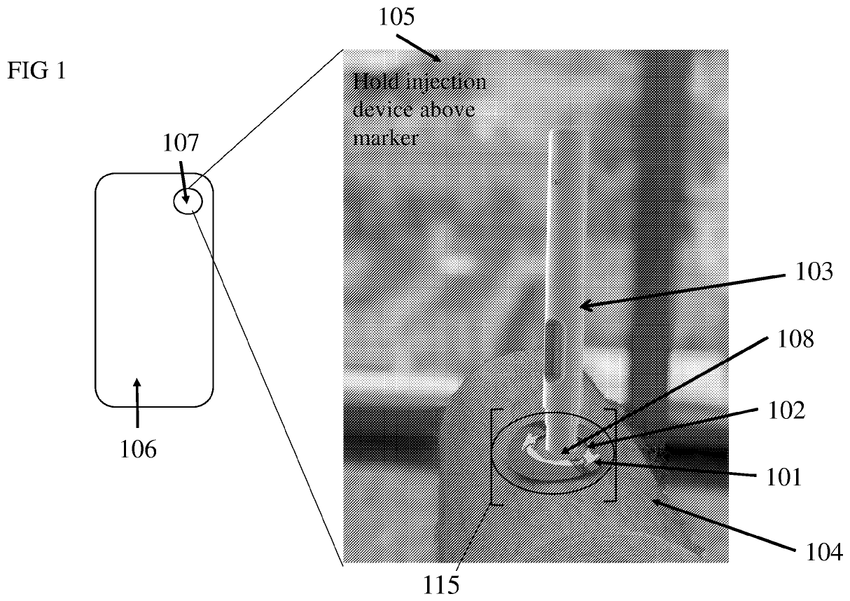

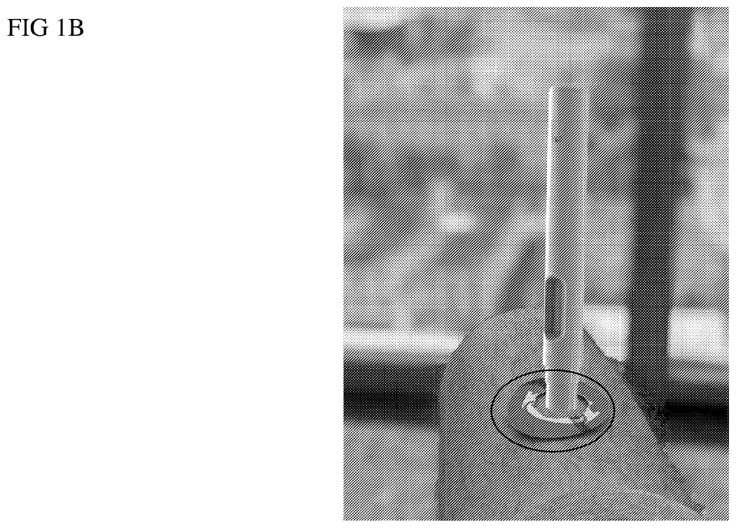

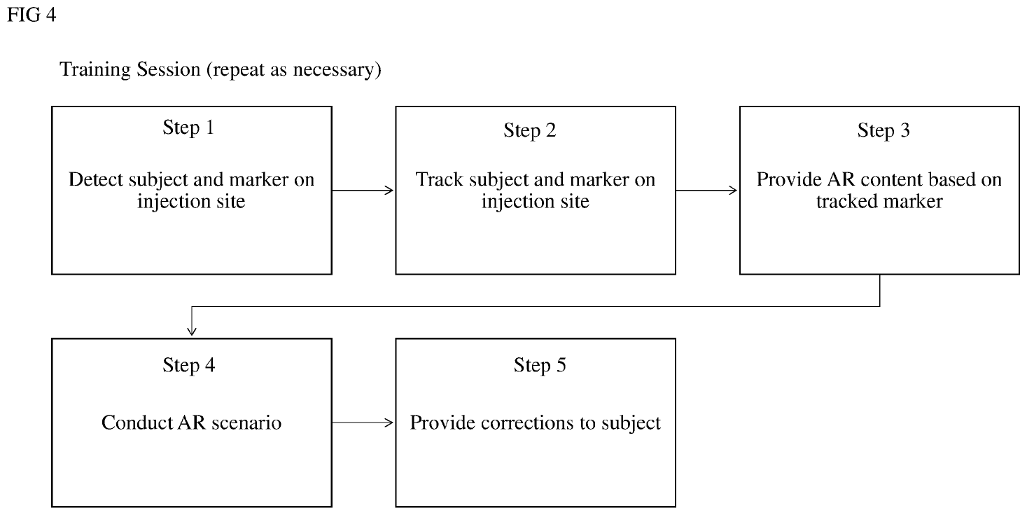

FIGS. 1,2, and 3 illustrate system embodiments, and FIG. 4 is a flow diagram of exemplary steps taken by the training system.

FIG. 1 shows the marker 101 on an injection site 108 and the AR content 103, for example, a virtual injection device displayed on a screen as shown, anchored to the marker 101. Those skilled in the art will appreciate that the term “anchor” and other grammatical forms thereof would have its normal meaning in the field of augmented reality. Typically, anchoring the AR content to the marker involves scaling the size of the AR content 103 based on the distance from the AR component to the marker 101. In certain embodiments, the injection site 108 is an intramuscular site or a subcutaneous site. The intramuscular injection site includes, but is not limited to, the deltoid, the vastus lateralis, the rectus femoris, the ventrogluteal, or the dorsogluteal. For self-injection training, the self-injection sites include the deltoid, the vastus lateralis, or the rectus femoris.

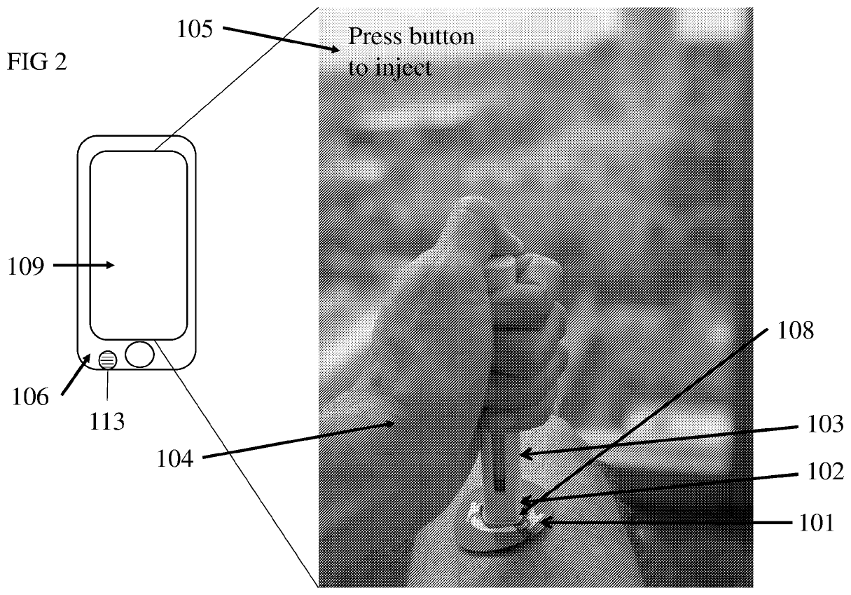

As seen in FIG. 1, the marker 101 is adhered to the injection site 108 and contains an opening 102 to admit an injection device 110. The marker 101 is a visual cue that the AR component 106 tracks to display the AR content 103 and anchor the AR content 103 to the marker 101. The marker 101 comprises an opening 102 for receiving the injection device 110 and an attaching side. The AR content 103 can be any virtual representation of any commercially available injector system that is provided via the AR component 106, such as on a screen as shown in FIG. 2. The most common injector systems commercially available are pen injectors, syringes, and autoinjectors. Demo

[symbol]

The AR component 106 may take several forms, but is typically a device that includes a processor, display screen, sensors (e.g. camera, microphone), speaker and, optionally. input devices. Examples of AR components include heads up displays (HUD), holographic displays, smart glasses, handhelds (e.g. tablets or smartphones), or computers. Smart glasses include Oculus Quests, Microsoft HoloLens, or Google Glasses. Handhelds include tablets or smartphones. In a specific embodiment, the AR component 106 comprises a smartphone. In an exemplary embodiment illustrated in FIG. 1, the device is equipped with a camera 107. The AR component 106 may be programmed to provide a virtual overlay 115 on the screen 109 (see FIG. 2) to assist the user to adjust the alignment or distance of the AR component 106 relative to the marker 101 and/or the subject 104. In addition, the AR component 103 may be programmed to provide animation on the screen 109 that includes visual imagery of proper operation or usage of the AR content. Proper usage of the AR content may also include steps that occur prior to a simulated injection, such as but not limited to, medicament reconstitution, for example checking medicament fluid for proper mixing and absence of bubbles in a medicament fluid.

The AR component 106 is programmed to detect and track the subject 104 and marker 101 using the camera 107. One skilled in the art would appreciate the multiple methods available for programming the AR component 106 to detect and track the marker. In alternative embodiments, the AR component 106 may detect the marker 101 by an RFID sensor, wireless communication, near-field communication, Bluetooth, or a matrix barcode, wherein the marker 101 includes necessary circuitry/components to execute such detection methods.

FIG. 2 illustrates an exemplary embodiment of the system as viewed through the screen 109 of the AR component 106. In addition, the AR component 103 may be programmed to display text 105 on the screen 109 to provide guidance and other information to user and/or narrated instructions concerning the AR content may be outputted via speaker 113 that provides instructions for using the injection device in a sequence of steps. corrections of error conditions, or any feedback to the subject. Error conditions may include, in non-limiting examples, putting cap back on a device, an out of sequence operation of a device, a wet injection, and not holding at 90 degrees when required.

The subject 104 uses the AR component 106 to perform a virtual injection using the AR content 103, which is shown in FIGS. 1-3 as an autoinjector. In a specific embodiment, the subject 104 performs a virtual self-injection using the AR content 103 at the injection site 108 located within the opening 102 of the marker 101. The AR component 106 tracks the subject 104 during the AR scenario and adjusts the AR content 103 in real-time based on the subject's actions. This can include moving the position of the virtual injection device based on the subject's movements, e.g., when the subject moves their hand holding the AR content 103 in the AR scenario. The subject may repeat the training until they feel comfortable to perform a physical injection.

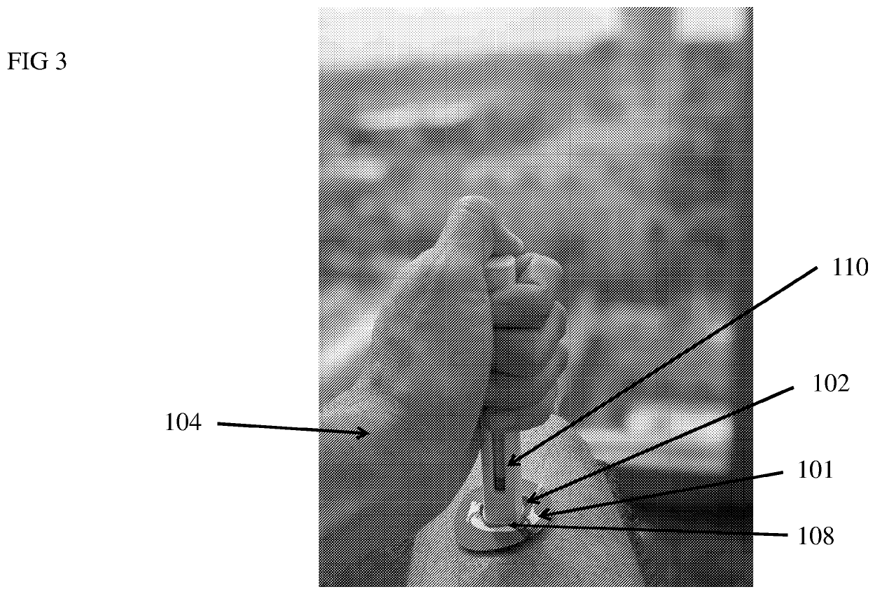

As shown in FIG. 3, the system provides guidance for self-administration of a physical injection device 110. After a subject 104 has completed training using the AR injection training system. the subject 104 can perform an actual physical injection using the marker. The marker 101 may be left on the injection site 108 post AR training. The subject 104 can administer an injection 110 using the marker 101 as a guide for the injection site 108. If the subject needs to administer multiple injections at a second injection site, the marker may be detached and adhered to the second injection site. In certain embodiments, the marker 101 is made of a flexible material (e.g. rubber, silicone, etc.) and optionally comprises a friction surface (not shown) that allows the user to pinch the edges of the marker 101 so as to pinch the underlying tissue of the injection site 108 during actual injection. In an alternative embodiment, the marker 101 is made of a stiff material that allows the user 104 to flatten out tissue around the injection site 108 by depressing down on the marker 101.

Turning to FIG. 4, a flow diagram is presented representing exemplary steps taken by the training system. In step 1, the AR component detects the marker on an injection site and detects the subject performing the training. In certain embodiments, the marker is detected by a camera associated with the AR component. Step 2 involves the AR component tracking the marker and the subject's movements continuously throughout the training. The AR scenario is begun in Step 3 with the AR component anchoring AR content (e.g. virtual autoinjector) to the marker. In Step 4, the AR component conducts the training with the subject performing a virtual injection using the AR content. Step 4 can be repeated as necessary for the subject's comfort. Step 5 can be performed during or after Step 4, and involves correcting errors made during the training. Errors may include, in non-limiting examples, putting cap back on a device, an out of sequence operation of a device, a wet injection, and not holding at 90 degrees when required. The correction may be given as text in the AR content or as an AR scenario showing the correction. In a specific embodiment, the AR content shows proper methods for correcting the error.

It is also contemplated that the AR component 106 can be programmed to facilitate use of an actual physical device such as an injection device or a trainer device (containing no medicine). For example, the AR component may be programmed to provide a virtual overlay on the screen 109 such as text, images or animations that are displayed to direct the user on proper use of the physical device.

Often, after using a particular injection device users may need to switch brands of drug wherein the subsequent brand is delivered using a different physical device. In another embodiment, the AR component 106 is programmed to provide virtual imagery (e.g. images, annimations, text) on the screen 109 and/or auditory information through speaker 113 that explain differences in operation between two different devices.

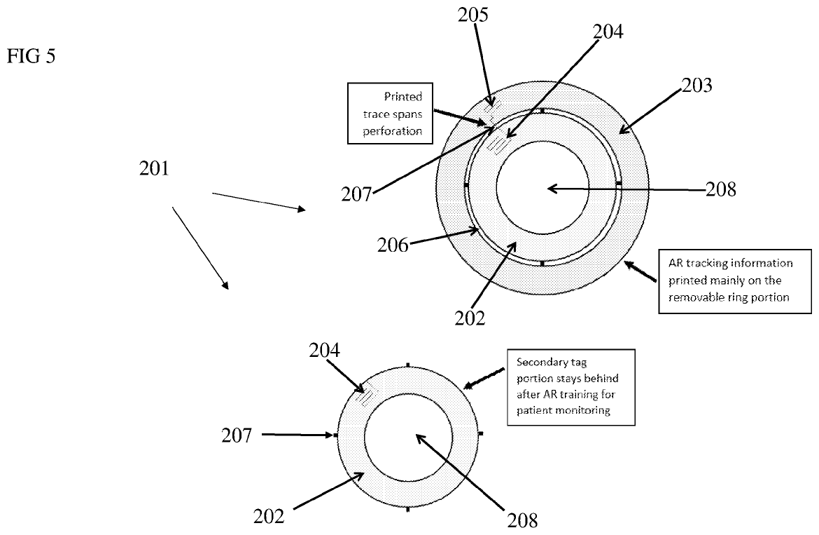

In certain embodiments, the marker has one or more medical sensors for monitoring of vital signs or other health indicators in the subject. FIG. 5 depicts an embodiment of a marker 201 with integrated medical sensors 204,205. The medical sensor 204,205 can detect placement on skin, temperature, strain (potentially indicative of activation), acceleration (multi-axis), LED transmissibility (pulse-ox detection, heart rate, etc.), or removal of the marker. The marker 201 contains an opening 208 to provide throughwhich AR and/or actual injections are executed. In a specific embodiment, the marker 201 has at least two portions 202 and 203 and has a perforated section 206 to facilitate separation of portions 202, 203 to leave limited sensors 204 in contact after AR training (FIG. 5). The AR tracking information is located on a removable portion 203 of the marker 201, and once removed. a secondary portion 202 remains for patient monitoring and performing injections. The removal along the perforations 206 can be detected by the medical sensor 204,205 by incorporating traces 207 through the perforation.

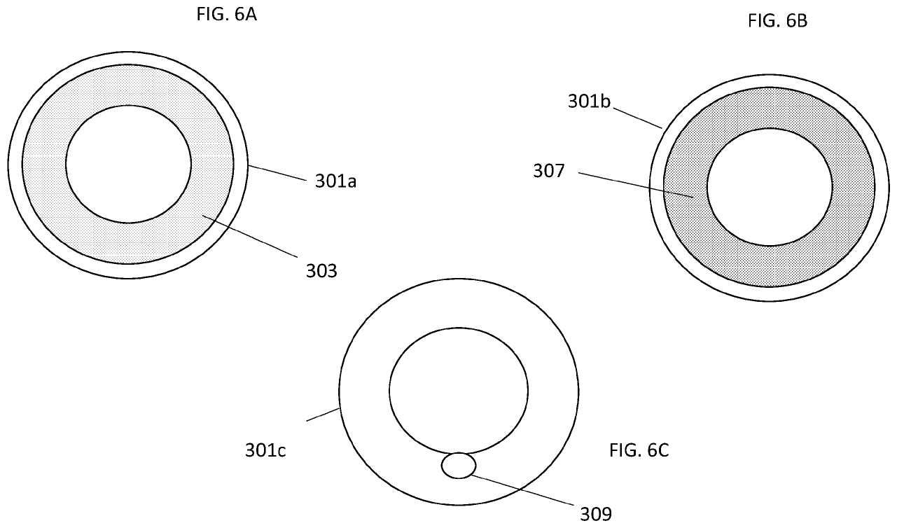

In a specific embodiment as shown in FIG. 6A, the attaching side of the marker 301 is shown which contains analgesic and/or anesthetic medications 303 on the attaching side of the marker 301a. The medications are topically released by the marker to the skin around the injection site. The medications numb the area or reduce pain prior to and following the injection. Examples of analgesic and/or anesthetic medications include, but are not limited to, benzocaine, butamben, dibucaine, lidocaine, oxybuprocaine, pramoxine, proxymetacaine, and tetracaine. Alternatively, as shown in FIG. 6B, the marker 301b may be equipped with a cold producing component 307 such as lining or pouch located on the attaching side of the marker 101 with chemicals for inducing an endothermic reaction contained within at least one breakable compartment. Upon breaking the compartment. the chemicals combine to produce an endothermic reaction. Examples of endothermic reagents include. for example, an ammonium salt (e.g. ammonium chloride or ammonium nitrate) and water. The production of cold serves as a natural analgesic. In another example as shown in FIG. 6C, a vibration component 309 is provided on the marker 301c (attaching side or opposite side) wherein vibration is initiated prior to and/or during an actual injection event. Vibration can serve as an pain-reliever and/or distraction. Any combination of the features described above in relation to FIGS. 6A-C is contemplated.

The combination of features depicted in the Figures is not intended to be limiting. For example, where a combination of features are shown and described, versions of embodiments that only include one or smaller combination of the described or depicted features are also included and contemplated as embodiments.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. An augmented reality (AR) injection training system for training a subject to administer injections, the system comprising: an injection site marker, an AR component, and optionally an physical device for which training occurs.

2. The system of claim 1, wherein the marker comprises an attaching side that abuts tissue of the subject.

3. The system of claim 2, wherein the attaching side comprises an adhesive layer.

4. The system of claim 1, wherein the marker can be attached to an intended injection site on the subject.

5. The system of any of claims 1-4, wherein, the marker comprises an opening to admit the injection device.

6. The system of any of claims 1-5, wherein the AR component comprises a computing device comprising a camera and screen.

7. The system of claim 6, wherein the AR component is configured to display a virtual image overlay on the screen that facilitates establishing a proper viewing distance from the injection site.

8. The system of claim 7, wherein alignment of the virtual image overlay with the injection site marker, initiates provision of training content by the AR component.

9. The system of any of claims 1-8, wherein the AR component is configured to detect the injection site marker and the subject, when the marker is positioned on an injection site on the subject.

10. The system of claim 6, wherein the AR component tracks the injection site marker and the subject via the camera.

11. The system of any of claims 6-10, wherein the AR component anchors AR content to the marker on the screen.

12. The system of any of claims 1-11, wherein the AR content comprises a virtual injection device, and optionally, the AR component is programmed to provide audio content mimicking sounds that occur during injection.

13. The system of claim 11, wherein the AR component is programmed to provide animation of proper operation or usage of the AR content.

14. The system of claim 10, wherein the AR component is programmed to provide a virtual overlay on the screen to provide images onto a physical device that provide instructions on usage of the physical device, wherein the physical device is an actual injection device or a trainer device.

15. The system of claims 10-14, wherein the AR component is programmed to provide written instructions on the screen and/or narrated instructions via a speaker directed to operation of the AR content.

16. The system of claim 1, wherein the subject uses the AR component to train for self-injection.

17. The system of claim 13, wherein the AR component provides visual and/or audio feedback to the subject to direct corrections on the subject's training.

18. The system of any of claims 1-17, wherein the AR component provides visual and/or auditory feedback to the subject during or after training to correct errors made while training.

19. The system of claim 18, wherein the error conditions comprise putting cap back on a device, an out of sequence operation of a device, a wet injection, or incorrect position of a device.

20. The system of claim 1, the physical device comprises a syringe injector, a pen injector, autoinjector, or an electronic injection device and training devices related to same.

21. The system of claim 1, wherein the marker further comprises one or more medical sensors.

22. The system of any of claims 1-20, wherein the AR component is programmed to provide virtual images that instruct the user on differences in function and/or operation of two different physical devices.

23. The system of claim 21, wherein the medical sensor detects marker location, temperature, strain, acceleration, and/or LED transmissibility.

24. The system of any of claims 1-23, wherein the attaching side comprises an analgesic or anesthetic medication, a cold component and/or a vibration component.

25. The system of claim 24, wherein the analgesic or anesthetic medication comprises benzocaine, butamben, dibucaine, lidocaine, oxybuprocaine, pramoxine, proxymetacaine, or tetracaine.

26. The system of any of claims 1-25, wherein the marker comprises a removable section and a remaining section.

27. The system of any of claims 1-26, wherein the marker is made of a flexible material so as to facilitate pinching of the edges of the marker so as to pinch the underlying tissue.

28. The system of any of claim 1-26, wherein the marker is rigid so as to facilitate flattening underlying tissue of the user upon depression of the marker on the subject.

29. A method for using an AR injection training system by a subject in need comprising attaching a marker to an injection site, using an AR component to detect the marker and the subject, conducting a virtual administration via the AR component, optionally receiving corrections from the AR component, and optionally administering an injection device to the injection site indicated by the marker.

30. The method of claim 29, wherein the AR component is configured to provide narrated instructions to the user during the conducting step.

31. The method of claim 30, wherein the narrated instructions comprise information about the operation of a physical device.

32. The method of any of claims 29-31, wherein, the marker comprises an opening to admit the injection device.

33. The method of claim 29, wherein the injection site comprises a site for subcutaneous or intramuscular injection.

34. The method of any of claims 29-33, wherein the marker can be removed from a first injection site and placed at a second injection site.

35. The method of claim 29, wherein the injection sites are located on the subject in need.

36. The method of claim 35, wherein the subject in need performs self-injections or self-administrations.

37. The method of claim 29, wherein the AR component comprises a smartphone, computer, a tablet, a virtual reality device, or any device with a camera and a screen.

38. The method of claim 29, wherein the injection device comprises a syringe injector, a pen injector, autoinjector, or an electronic injection device or a training device of any of the foregoing.

39. A marker for use with an AR component, the marker comprising peripheral section and an opening defined within the peripheral section, an adhesive layer disposed on a side of the marker.

40. The marker of claim 39, wherein the marker further comprises one or more medical sensors.

41. The marker of claim 40, wherein the medical sensor detects marker location, temperature, strain, acceleration, and/or LED transmissibility.

42. The marker of claim 39, 40 or 41, wherein the attaching side comprises an analgesic or anesthetic medication, a cold component and/or a vibration component.

43. The marker of claim 42, wherein the analgesic or anesthetic medication comprises benzocaine, butamben, dibucaine, lidocaine, oxybuprocaine, pramoxine, proxymetacaine, or tetracaine.

44. The marker of any of claims 39-43, wherein the marker comprises a removable section and a remaining section.

45. The marker of any of claims 39-44, wherein the marker is made of a flexible material so as to facilitate pinching of the edges of the marker so as to pinch the underlying tissue.

46. The marker of any of claims 39-44, wherein the marker is rigid so as to facilitate flattening underlying tissue of the user upon depression of the marker on the subject.
